(19) United States
(12) Patent Application Publication
(10) Pub. No.: US 2014/0316236 A1
(43) Pub. Date: Oct. 23, 2014

(54) OBJECT INFORMATION ACQUIRING APPARATUS AND CONTROL METHOD FOR OBJECT INFORMATION ACQUIRING APPARATUS

(71) Applicant: CANON KABUSHIKI KAISHA, Tokyo (JP)

(72) Inventor: Kohtaro Umezawa, Kyoto-shi (JP)

(73) Assignee: CANON KABUSHIKI KAISHA, Tokyo (JP)

(21) Appl. No.: 14/245,039

(22) Filed: Apr. 4, 2014

(30) Foreign Application Priority Data
Apr. 17, 2013 (JP) 2013-086694

(51) Int. Cl. A61B 5/11; A61B 5/00

(52) U.S. Cl. CPC A61B 5/1128; A61B 5/0095; A61B 5/742
USPC 600/407

(57) ABSTRACT

An object information acquiring apparatus comprises an acoustic wave probe that receives an acoustic wave arriving from an interior of an object; a position information acquisition unit that acquires position information which is information on a position of the object; and a notification unit that notifies an operator of a change in the position of the object, based on the position information.

### BACKGROUND OF THE INVENTION

1. Field of the Invention

The present invention relates to an object information acquiring apparatus that acquires information inside an object, and a control method thereof.

2. Description of the Related Art

A photoacoustic imaging apparatus that images the state and functions inside a tissue by irradiating light, such as laser light, onto an organism and receiving an ultrasound wave generated from inside the organism due to this, is frequently used in medical fields. If measurement light, such as a pulsed laser light, is irradiated onto an object, an acoustic wave is generated when the measurement light is absorbed by the biological tissue inside the object. The photoacoustic imaging apparatus receives the generated acoustic wave using a probe and analyzes the acoustic wave, whereby information related to the optical characteristic (functional information) inside the object can be visualized. This technique is called “photoacoustic imaging”.

To acquire an ultrasound wave over a wide range, an image diagnostic apparatus that includes a mechanism for a probe to mechanically scan an object has been proposed. For example, Japanese Patent Application Laid-open No. 2010-104816 discloses a photoacoustic imaging apparatus that can acquire ultrasound waves over a wide range by allowing a probe to mechanically scan an object.

### SUMMARY OF THE INVENTION

In the above mentioned photoacoustic imaging apparatus, the probe moves on the surface of an object for scanning. Therefore if the object moves during scanning, a shift may be generated in the acquired images or data to be acquired may not be acquired. Even in a case of a non-scanning type photoacoustic imaging apparatus, a correct image cannot be acquired if the object moves during measurement, since the image is constructed by integrating the acoustic wave generated from the object for a predetermined time.

Thus in an apparatus that acquires information on an object using an acoustic wave, care must be taken so that the object does not move during measurement. If movement of the object during measurement is recognized after the measurement, it is necessary to redo the measurement from the beginning while compressing and holding the object, which is a huge burden on the testee.

With the foregoing in view, it is an object of the present invention to provide an object information acquiring apparatus that can notify the operator of a shift in the position of the object during the measurement.

The present invention in its one aspect provides an object information acquiring apparatus comprises an acoustic wave probe that receives an acoustic wave arriving from an interior of an object; a position information acquisition unit that acquires position information which is information on a position of the object; and a notification unit that notifies an operator of a change in the position of the object, based on the position information.

The present invention in its another aspect provides a control method for an object information acquiring apparatus having an acoustic wave probe that receives an acoustic wave arriving from an interior of an object, the method comprises a reception step of receiving an acoustic wave in use of the acoustic wave probe; a position information acquisition step of acquiring position information which is information on a position of the object; and a notification step of notifying an operator of a change in the position of the object, based on the position information.

According to the present invention, an object information acquiring apparatus that can notify an operator of a shift in the position of the object during the measurement can be provided.

Further features of the present invention will become apparent from the following description of exemplary embodiments with reference to the attached drawings.

### BRIEF DESCRIPTION OF THE DRAWINGS

FIG. 1 is a diagram depicting a configuration of a photoacoustic measurement apparatus according to Embodiment 1;

FIG. 2 is a flow chart depicting an operation of the photoacoustic measurement apparatus according to Embodiment 1;

FIG. 3 is a diagram for describing an operation console of the photoacoustic measurement apparatus according to Embodiment 1;

FIG. 4 is a diagram for describing an operation timing of each composing element of the photoacoustic measurement apparatus;

FIG. 5 is a diagram for describing an operation console of a photoacoustic measurement apparatus according to a modification;

FIG. 6 is a diagram depicting a configuration of a photoacoustic measurement apparatus according to Embodiment 2;

FIG. 7 is a flow chart depicting an operation of the photoacoustic measurement apparatus according to Embodiment 2; and

FIG. 8 is a diagram for describing an operation console of a photoacoustic measurement apparatus according to Embodiment 3.

### DESCRIPTION OF THE EMBODIMENTS

Embodiments of the present invention will now be described with reference to the drawings. As a rule, the same composing elements are denoted with a same reference number, and redundant description is omitted.

### Embodiment 1

A photoacoustic measurement apparatus according to Embodiment 1 of the present invention is an apparatus that images optical characteristic value information inside an object by irradiating laser light onto an object, and receiving and analyzing a photoacoustic wave generated inside the object due to the laser light. The optical characteristic value information is normally initial sound pressure distribution, light absorption energy density distribution, absorption coefficient distribution or concentration distribution of substance constituting a tissue.

<System Configuration>

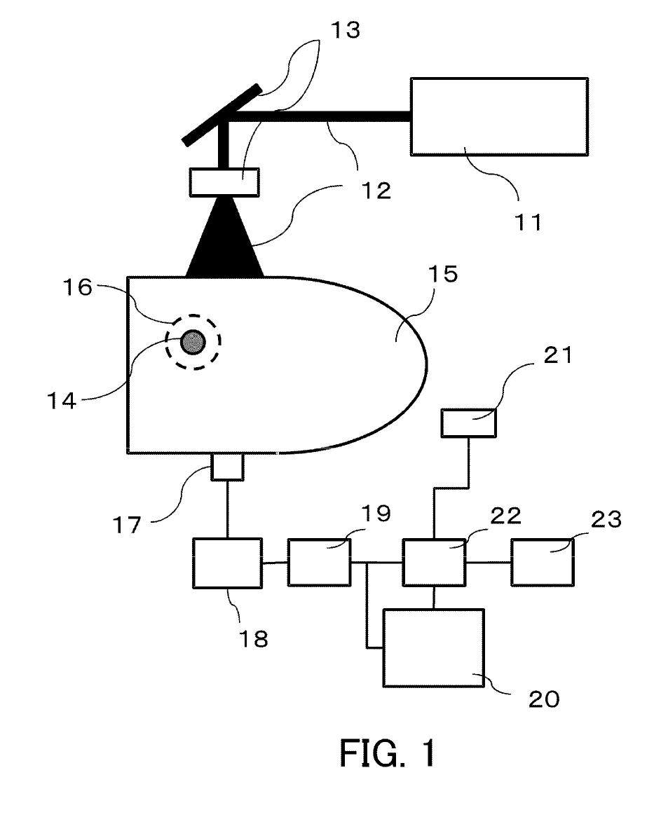

A configuration of the photoacoustic measurement apparatus according to Embodiment 1 will be described with reference to FIG. 1. The photoacoustic measurement apparatus according to Embodiment 1 includes a light source 11, an optical system 13, an acoustic wave probe 17, a signal processing unit 18, a data processing unit 19, an input/output unit 20, a measurement unit 21, a change detection unit 22, and a notification unit 23.

Measurement is performed in a state where an object 15 (e.g. breast) is inserted into an opening (not illustrated) created in the apparatus.

First a pulsed light 12 emitted from the light source 11 is irradiated onto the object 15 via the optical system 13. When a part of the energy of the light that propagates inside the object is absorbed by a light absorber, such as blood, an acoustic wave 16 is generated from the light absorber by the thermal expansion. The acoustic wave generated inside the object is received by the acoustic wave probe 17, and is analyzed by the signal processing unit 18 and the data processing unit 19. The analysis result is converted into image data representing the characteristic information inside the object (optical characteristic value information data), and is outputted through the input/output unit 20.

In the photoacoustic measurement apparatus of this embodiment, if the measurement unit 21 acquires information to indicate a position of the object (position information) and this information indicates a generation of a position change of the object that influences the measurement, the change detection unit 22 detects this change, and notifies the operator of this state using the notification unit 23. Thereby the operator can recognize that the position of the object changed during measurement (that is, know that re-measurement or the like is required).

Each unit constituting the photoacoustic measurement apparatus according to the present embodiment will now be described.

<<Light source 11>>

The light source 11 generates pulsed light that is irradiated onto an object. The light source is preferably a laser light source in order to obtain high power, but a light emitting diode, a flash lamp or the like may be used instead of a laser. If a laser is used for the light source, various lasers including a solid-state laser, a gas laser, a dye laser and a semiconductor laser can be used. Irradiation timing, waveform, intensity or the like are controlled by a light source control unit (not illustrated). This light source control unit may be integrated with the light source.

To effectively generate a photoacoustic wave, light must be irradiated for a sufficiently short period of time in accordance with the thermal characteristics of the object. If the object is an organism, the pulse width of the pulsed light generated from the light source is preferably about 10 to 50 nanoseconds. The wavelength of the pulsed light is preferably a wavelength which allows the light to propagate inside the object. In concrete terms, a wavelength of 500 nm or more and 1200 nm or less is preferable if the object is an organism. Further, it is preferable to choose a wavelength of the pulsed light of which absorption coefficient, with respect to the observation object, is high.

<<Optical system 13>>

The optical system 13 guides the pulsed light 12 generated in the light source 11 to the object 15, and is typically constituted by, for example, a mirror that reflects light, a lens that collects, expands or changes the shape of light, and a diffusion plate that diffuses light. Using these optical elements, the irradiation conditions of the pulsed light, including irradiation shape, light density and irradiation direction to the object, can be freely set. It is preferable that the light is spread over a certain sized area rather than condensed by a lens, from the viewpoint of issues regarding the safety of the object and broadening of a diagnosis area. The light source 11 and the optical system 13 correspond to the light irradiation unit of the present invention.

<<Object 15>>

The object 15 and the light absorber 14 are not composing elements of the present invention, but will be described hereinbelow. The object 15 is a target of the photoacoustic measurement and typically is a breast, finger, limb or the like of a human or animal. Here it is assumed that the object is a human breast.

In the photoacoustic measurement apparatus according to this embodiment, a light absorber 14 having a relatively large light absorption coefficient existing inside the object 15 can be imaged. If the object is an organism, the light absorber 14 is, for example, water, lipids, melanin, collagen, protein, oxyhemoglobin or deoxyhemoglobin. A light absorber 14 may also be blood vessels containing a large quantity of oxyhemoglobin or deoxyhemoglobin, or a malignant tumor that includes many angiogenic blood vessels. By imaging a light absorber, the photoacoustic measurement apparatus according to this embodiment can preform angiography, diagnosis of malignant tumors and vascular diseases of humans and animals, and follow up observation of chemotherapy.

<<Acoustic Wave Probe 17>>

The acoustic wave probe 17 receives an acoustic wave generated inside the object due to the light irradiated onto the object 15, and converts the acoustic wave into an analog electric signal. The acoustic wave in the present invention is typically an ultrasound wave, including an elastic wave such as a sound wave, an ultrasound wave, a photoacoustic wave, and a light-induced ultrasound wave. The acoustic wave probe 17 receives such an elastic wave generated or reflected inside the object.

The acoustic wave probe 17 is also called a “probe” or a “transducer”. The acoustic wave probe 17 may be a standalone acoustic detector or may be constituted by a plurality of acoustic detectors. The acoustic wave probe 17 may be a plurality of reception elements which are arrayed one dimensionally or two dimensionally. If multi-dimensional array elements are used, the measurement time can be decreased since the acoustic wave can be received at a plurality of locations simultaneously, and can also reduce, for example, the influence of the vibration of the object.

It is preferable that the acoustic wave probe 17 has high sensitivity and a wide frequency band. In concrete terms, piezoelectric ceramics (PZT), polyvinylidene fluoride resin (PVDF), capacitive micro-machine ultrasonic transducer (CMUT), and a Fabry-Perot interferometer or the like can be used. The acoustic wave probe 17 is not limited to the examples mentioned here, but can be any material/component as long as the functions of an acoustic wave probe are satisfied.

<<Signal Processing Unit 18>>

The signal processing unit 18 amplifies an electric signal acquired by the acoustic wave probe 17, and converts the electric signal into a digital signal. The signal processing unit 18 is typically constituted by an amplifier, an A/D converter, a field programmable gate array (FPGA) chip and the like. If a plurality of detection signals are acquired from the probe, it is preferable that the signal processing unit 18 can process a plurality of signals simultaneously.

<<Data Processing Unit 19>>

The data processing unit 19 generates image data (reconstructs an image) by processing a digital signal acquired by the signal processing unit 18. The image reconstruction method that the data processing unit 19 executes is, for example, Fourier transform, universal back projection, filtered back projection, and sequential image reconstruction or the like, but any image reconstruction method can be used. The signal processing unit 18 and the data processing unit 19 may be integrated. The signal processing unit 18 and the data processing unit 19 correspond to the image acquisition unit in the present invention.

<<Input/Output Unit 20>>

The input/output unit 20 outputs an image generated by the data processing unit 19, and receives an input operation from an operator, and is a touch panel display in the case of this embodiment. The input/output unit 20 also displays detailed information on a position shift of an object if the later mentioned change detection unit 22 detects a position shift. The input/output unit 20 need not always be integrated with the photoacoustic measurement apparatus, but may be an apparatus connected externally.

<<Measurement Unit 21>>

The measurement unit 21 acquires position information of an object, and is, in concrete terms, a visible light camera or an infrared camera which images the surface of the object, or a distance sensor for measuring the shape of the object. If a camera is used for the measurement unit 21, the frame rate and resolution thereof may be high enough to detect a position shift of the object, which influences the measurement. The measurement unit 21 corresponds to the position information acquisition unit in the present invention.

The measurement unit 21 may be a plurality of visible light cameras or one or more sensor(s) that can measure the distance to the object. Any measurement unit may be used if the movement or deformation of the object can be detected, such as an infrared camera that can measure the shapes of blood vessels on the surface of the object.

In Embodiment 1, a visible light camera, that can capture the entire measurement target area of the object, is used as the measurement unit 21.

<<Change Detection Unit 22>>

The change detection unit 22 detects a shift of an object that occurs during the photoacoustic measurement based on the object image acquired by the measurement unit 21.

Here the shift of an object will be described. In the photoacoustic measurement, an acoustic wave generated inside the object is received by the probe, whereby a generation source of the acoustic wave is estimated. In other words, if the object moves or deforms during the photoacoustic measurement, the positional relationship of the object with respect to the probe changes, and an image is generated based on incorrect information. The change detection unit 22 detects such a shift of the object which influences the measurement (hereafter simply referred to as “position shift”). The position shift includes parallel movement, expansion/contraction, rotation, distortion and the like of the object in the measurement target area. Any movement of the object which influences the measurement can be detected here.

The change detection unit 22 acquires a plurality of object images using the measurement unit 21, and detects the generation of a position shift using these images. A concrete method thereof will be described later.

The signal processing unit 18, the data processing unit 19, and the change detection unit 22 may be a computer constituted by a CPU, a main storage device and an auxiliary storage device, or may be hardware, such as a microcomputer and a custom-designed FPGA.

<<Notification Unit 23>>

The notification unit 23 is an interface for notifying the operator that the change detection unit 22 detected a position shift. The change detection unit 22 and the notification unit 23 correspond to the notification unit in the present invention. In this embodiment, the notification unit 23 is a lamp that can emit a plurality of colors of light (e.g. normally green, which turns red when a position shift occurs), but may display a message to inform an operator that a position shift occurred, including details on the position shift, on a display or display panel.

The notification unit 23 may notify the operator by sound, such as an alarm or melody, when a position shift occurs. The notification may be performed by any method as long as the operator can recognize that a position shift of the object occurred. The notification unit 23 need not always be integrated with the photoacoustic measurement apparatus, but may be an apparatus connected externally. The notification unit 23 may be integrated with the input/output unit 20.

<<Position Shift Detection Method>>

A method for the change detection unit 22 to detect a position shift of an object will be described next. In this example, a target region (region of interest) to detect the position shift is determined in an object image, and the position shift in this region is detected. The region of interest may be specified by the operator in advance, or may be automatically set by the apparatus.

Position shift is detected by comparing a template image and an object image that is periodically acquired during measurement. First an object image is acquired before starting measurement, and this image is temporarily stored as the template image. After the measurement starts an object image is periodically acquired at every predetermined time, and the template image and an object image in each frame are matched. In concrete terms, a zero-mean normalized cross-correlation (ZNCC), as shown in Expression (1), is calculated, and the change amount of the position of the object is determined.

In this embodiment, calculation based on ZNCC is performed, but another calculation method may be used if the change of the position of the object can be determined. For example, any method for determining the change of the position of the object, such as sum of squared difference (SSD) or sum of absolute difference (SAD) may be used. In this example, a region of interest is the entire object image, but if a region of interest is specified, the region of interest may be extracted from each image to do calculation performed thereon.

$$\begin{matrix}[ Math . 1 ] & \\ R = \frac{{\sum }_{j = 0}^{N - 1} {\sum }_{i = 0}^{M - 1} ( ( I ( i , j ) - {I}_{avg} ) ( T ( i , j ) - {T}_{avg} ) )}{\sqrt{{\sum }_{j = 0}^{N 1} {\sum }_{i = 0}^{M 1} {( I ( i , j ) - {I}_{avg} )}^{2} \times {\sum }_{j = 0}^{N 1} {\sum }_{i = 0}^{M 1} {( T ( i , j ) - {T}_{avg} )}^{2}}} & Expression ( 1 )\end{matrix}$$

Here M and N are a number of pixels in the X direction and in the Y direction in the X-Y coordinate system of each image. I (i,j) is a brightness value in a region of interest of the object image during the measurement, and I_{avg} is an average value of the brightness in this region of interest. T (_{i,j}) is a brightness value in a region of interest of the template image, and T_{avg} is an average value of the brightness in this region of interest.

Similarity R between the region of interest of the template image and the region of interest of the object image can be determined using Expression (1). The respective shift width in the X direction and Y direction can be acquired by matching the template image and the object image while shifting the coordinates, and acquiring the shift amount when the similarity is highest. This shift width is the moving amount of the object generated after the start of the measurement.

The change detection unit 22 acquires the respective shift width in the X direction and Y direction as described above, and determines whether a position shift of the object occurred based on these shift widths. The determination method will be described in detail later.

<<Processing Flow Chart>>

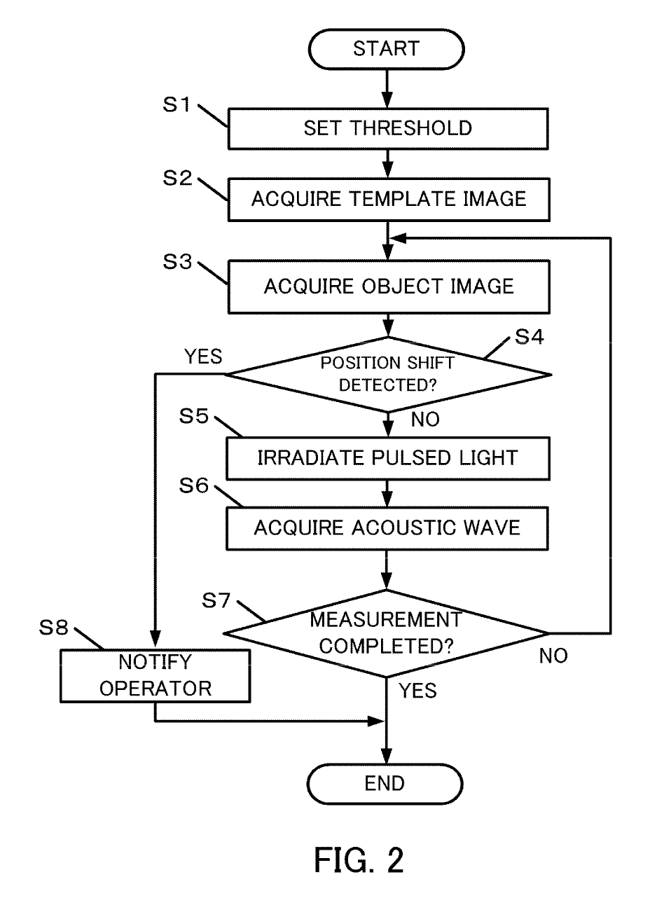

The processing executed by the photoacoustic measurement apparatus according to this embodiment will be described with reference to FIG. 2.

First the operator inputs a threshold of the shift width, which is an allowable maximum value of the moving amount, of the object using a number of pixels (S1). It is preferable to input the threshold via the input/output unit 20, but the threshold may be stored in the apparatus in advance as a predetermined value, or may be automatically calculated by the apparatus.

Then an object which is an organism (e.g. breast) is inserted into the photoacoustic measurement apparatus. At this time, the measurement unit 21 (visible light camera) captures an image of the object before and after the insertion of the object, and acquires the difference between these images. The difference of the images acquired here becomes a template image for comparison (S2). In the following description, an object image refers to a difference between an image captured in a state where the object is inserted and an image captured before the object is inserted (that is, an image of the object alone).

When step S2 ends, the photoacoustic measurement is started. First the measurement unit 21 acquires an object image (S3), and the change detection unit 22 detects the position shift of the object (S4). Here as mentioned above, the shift width between the object image acquired before the start of the measurement and each of the object images captured a plurality of times at every predetermined time during the measurement is acquired as a number of pixels, and compared with the predetermined threshold. If the shift width exceeds the threshold, it is determined that a position shift of the object occurred.

Alternately, the shift width from the state at the start of the measurement may be integrated every time an object image is acquired, and it may be determined that the position shift of the object occurred when the integrated shift width exceeded the threshold.

If the shift width is within the threshold as a result of executing step S4, the pulsed light is generated from the light source 11 and irradiated onto the object via the optical system 13 (S5).

Then an acoustic wave generated inside the object due to the pulsed light is acquired by the acoustic wave probe 17 (S6). When the pulsed light is emitted for a predetermined number of times and the acquisition of the acoustic wave completes, it is determined whether all the measurements completed (S7), and if completed, the processing ends. If not completed, the processing returns to step S3 and the object image is acquired again.

If the shift width exceeds a threshold as a result of executing step S4, the processing moves to step S8, and the operator is notified that the shift width exceeded the threshold via the input/output unit 20 and the notification unit 23.

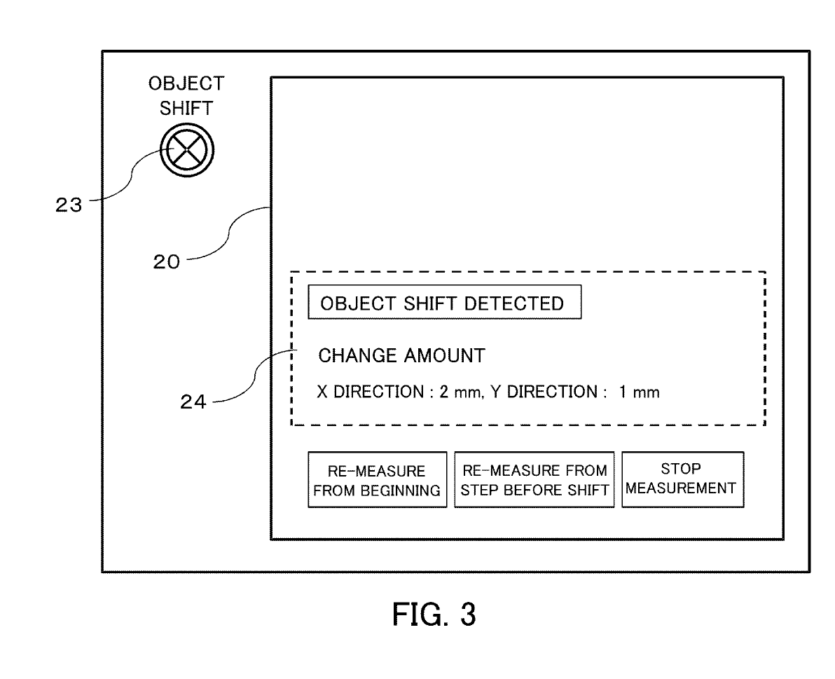

FIG. 3 is a diagram showing an operation console of the photoacoustic measurement device according to this embodiment. This operation console includes the input/output unit 20 (touch panel display) and the notification unit 23 (lamp). If the position shift of the object occurs, the lamp, which is normally lit green, changes to red, and detailed information (reference number 24) on the position shift is displayed on the touch panel display. In this embodiment, the detailed information is the positive change amount (number of changed pixels in the X direction and Y direction respectively) on the image of the object.

The position change amount of the object may be displayed as length (mm) or may be displayed as a changed voxel values or vector of the change amount. If a number of changed pixels in the Z direction can be detected, this information may also be displayed. The position change amount may be displayed in any format as long as the apparatus can process these values.

The object image before starting the measurement and the object image after the position shift occurred may be superimposed and displayed. A graphic to indicate the motion vector of the object may be generated and superimposed as well for display. Any display can be performed as long as the operator can be notified on how the object moved or deformed.

Options to select the subsequent processing are also displayed on the touch panel display. The content of the options can be any processing that the apparatus can execute, such as “Re-measure from beginning”, “Re-measure from step before shift” and “Stop measurement”.

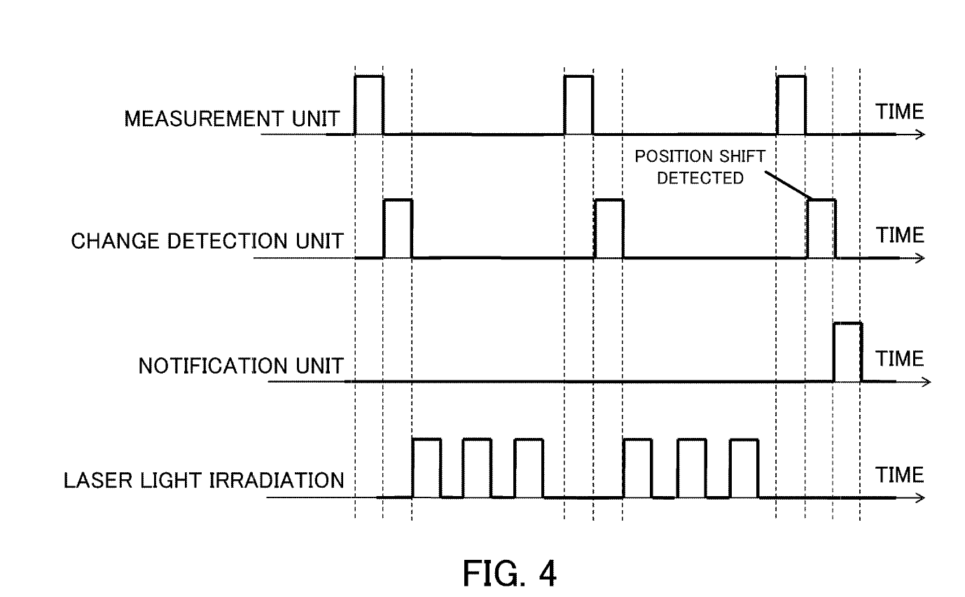

FIG. 4 is a diagram showing a relationship of the operation timings of the measurement unit 21, the change detection unit 22 and the notification unit 23 and the laser light irradiation timing. The measurement unit 21 acquires an object image and transmits the result to the change detection unit 22. The change detection unit 22 compares the template image and the acquired object image, and starts irradiation of the laser light if it is determined that a position shift did not occur. If it is determined that a position shift occurred, on the other hand, the change detection unit 22 notifies this state to the operation via the notification unit 23 without starting irradiation of the laser light.

According to Embodiment 1, in the photoacoustic measurement apparatus that images the acoustic wave generated from an object by integrating the acoustic wave for a predetermined time, a position shift of the object that occurred during the measurement can be accurately notified to the operator.

In this embodiment, the position shift of the object is detected by determining the ZNCC between the template image and an object image which is acquired at every predetermined time, but another method may be used. For example, a contour of the object image may be extracted from each frame, and the shift width may be calculated by mutually matching the contours.

A blood vessel image acquired by an infrared camera may be regarded as a template image, and the position shift of the object may be detected by calculating the ZNCC between frames. Further, the position shift of the object may be detected by comparing the center of gravity with the template image, or the position shift of the object may be detected by removing a template image in use of a region of interest set by the operator, and matching the region of interest with the object image.

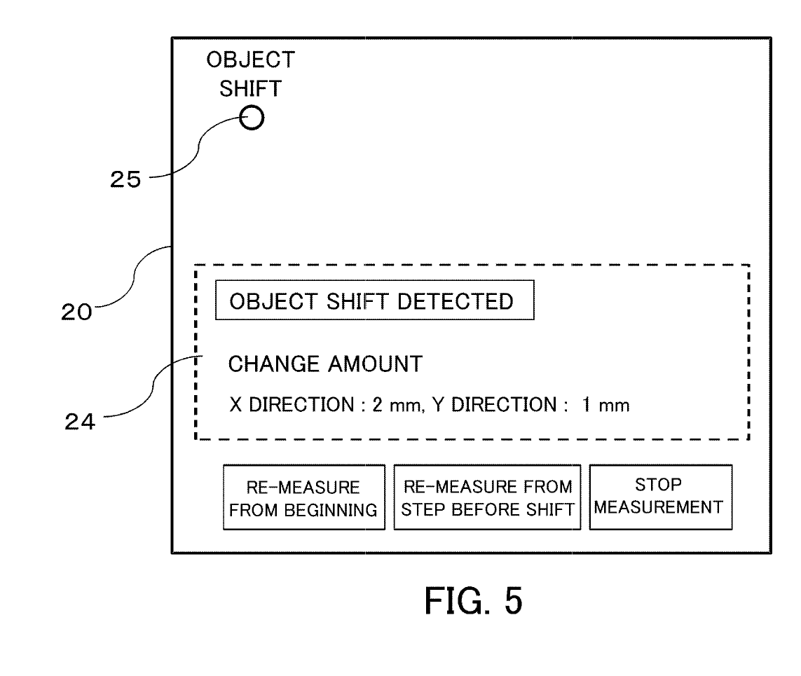

In this embodiment, a lamp is used as the notification unit 23, but the notification may be performed by graphics displayed on the display, as shown in FIG. 5, or the notification may be performed by sound using an acoustic apparatus. To perform notification using sound, an alarm or musical melody may be used. Any sound may be used as long as the operator can recognize the meaning of the notification.

### Embodiment 2

In Embodiment 1, the acoustic wave probe 17 is fixed with respect to the object 15. Whereas in Embodiment 2, the object is measured by mechanically scanning with the acoustic wave probe 17.

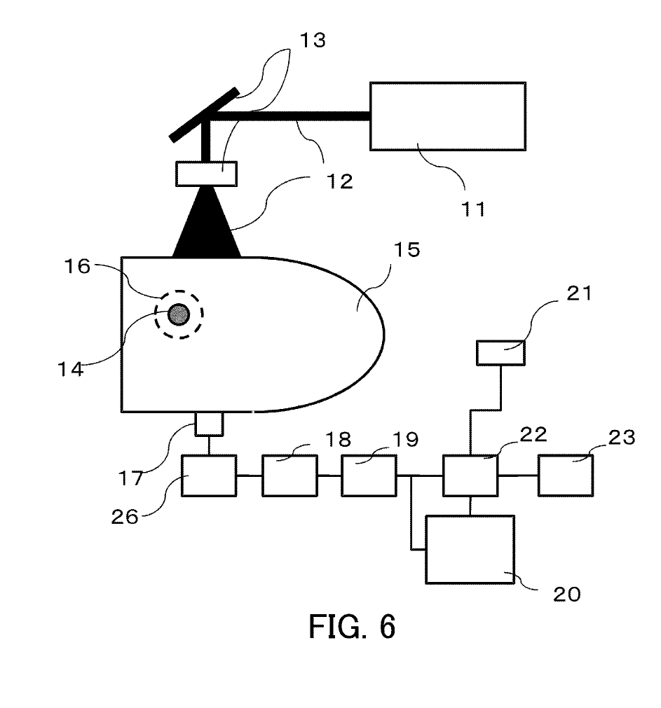

FIG. 6 shows the configuration of an ultrasonic diagnostic apparatus according to Embodiment 2. The configuration of the ultrasonic diagnostic apparatus according to Embodiment 2 is the same as Embodiment 1, except for a scanning unit 26 that scans with the acoustic wave probe 17 in two dimensional directions.

The scanning unit 26 moves the acoustic wave probe 17 in two dimensional directions, and is constituted by a scanning mechanism and a control unit thereof. By using the scanning unit 26, the photoacoustic measurement can be performed while allowing the acoustic wave probe 17 to scan two dimensionally. In this embodiment, the object 15 is fixed, and the relative positions of the object and the acoustic wave probe are changed by moving the acoustic wave probe on the X-Y stage.

In this embodiment, the acoustic wave probe 17 is moved using the scanning mechanism, but a configuration where the ultrasonic probe is fixed and the object is moved may be used. In this case, a support unit (not illustrated) to support the object may be moved using the scanning mechanism.

Further, both the object 15 and the acoustic wave probe 17 may be constructed to be movable. In the case of moving the object 15, it is preferable that the measurement unit 21 moves in the same way as the object by tracking the object, but same movement is not always necessary if the movement of the object can be detected. The scanning is preferably performed while moving the probe continuously, but may be performed while moving the probe intermittently. The scanning mechanism for scanning is preferably an electric type using a stepping motor or the like, but may be a manual scanning type.

The type of the scanning mechanism and the scanning method are not limited to those described in this example, but may be any mechanism or method only if at least one of the object 15 and the acoustic wave probe 17 can be moved.

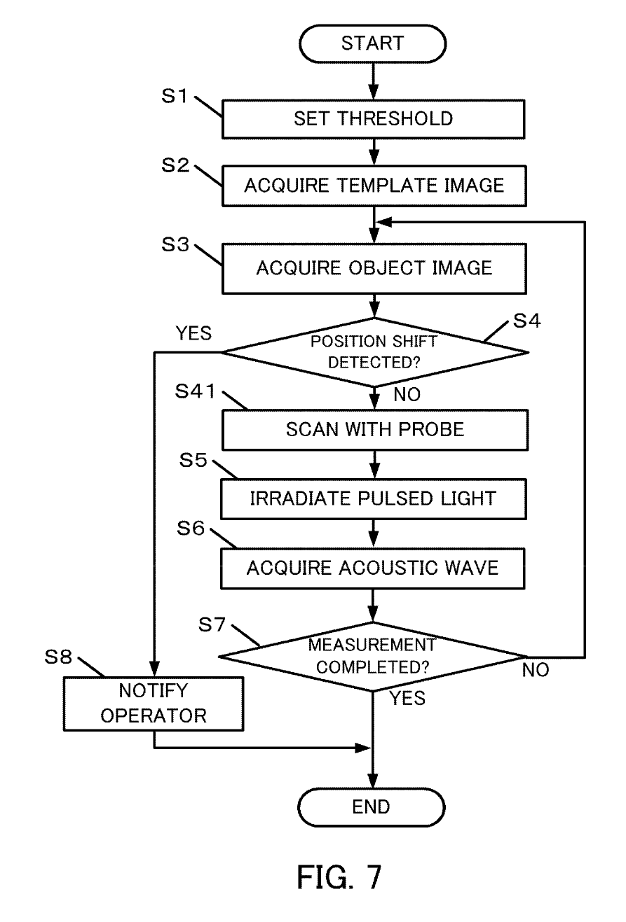

FIG. 7 shows a flow chart depicting the processing executed by the photoacoustic measurement apparatus according to Embodiment 2. The processing executed by the photoacoustic measurement apparatus according to Embodiment 2 is approximately the same as in Embodiment 1, but the difference is that step S41, where the scanning unit 26 moves the acoustic wave probe 17, is added before step S5, where the pulsed light is irradiated.

In this way, the present invention can also be applied to a photoacoustic measurement apparatus that performs measurement of the object by scanning with an acoustic probe.

### Embodiment 3

In Embodiment 1 and Embodiment 2, a shift width between the template image acquired before starting the photoacoustic measurement and an object image acquired during measurement is acquired. In other words, the position shift of the object is expressed as one vector. In Embodiment 3, on the other hand, feature points on the surface of the object are extracted and a displacement amount is determined for each feature point, whereby the generation of a position shift of the object depending on the position is comprehensively determined.

The configuration of the ultrasonic diagnostic apparatus according to Embodiment 3 is the same as that of Embodiment 2, but a difference from Embodiment 2 is that the measurement unit 21 is not constituted by a standard camera, but by a stereo camera which can acquire distance data.

The other difference from Embodiment 2 is that the change detection unit 22 according to Embodiment 3 determines whether the position shift occurred not by pattern matching of the captured images but by extracting the feature points from each image and detecting the movement of the extracted feature points.

Known techniques can be used to extract the feature points. For example, the feature points may be extracted from edge information that is acquired by filtering the images, or may be extracted from the features of the biological structure in the images (e.g. nipple of a breast, shadow of blood vessels, melanin pigmentation, contour of breast, wrinkles). The feature point extraction method is not especially limited if only the change of positions of the feature points can be tracked between frames.

The feature points may be extracted from information acquired by integrating this information between frames for a predetermined time, and averaging the integration result. Only a part of the images may be used or all of the images may be used to determine the feature points from each imaged frame. Further, the operator may set a region of interest using the input/output unit 20, and track the feature points within the region of interest.

A feature point is a micro area for tracking the movement of an object, and need not necessarily correspond to one pixel.

A flow chart depicting the processing executed by the photoacoustic measurement apparatus according to Embodiment 3 will be described, focusing on the differences from Embodiment 2.

In step S1, a threshold of a position shift of an object, which is an allowable maximum value of the shift width, is set, just like Embodiment 2, but in Embodiment 3, not a value corresponding to the moving amount of the entire object but an allowable maximum value of a deformation amount is set as the threshold. In concrete terms, “allowable maximum value of the shift width of a feature point of which displacement amount is greatest” is set as the threshold. The allowable value of the shift width may be inputted as a number of pixels, or may be inputted as a voxel converted value or a distance converted value.

The threshold may be set automatically. For example, displacement formation (e.g. displacement vector value and absolute value thereof) corresponding to each feature point may be acquired in a period after the object is inserted into the apparatus and measurement preparation becomes ready and before the measurement is started, and the displacement information multiplied by a predetermined value may be used as the threshold. These operations may be performed by the measurement unit 21 or by the change detection unit 22.

In step S2, coordinates of the feature points in a state before the start of the measurement is acquired instead of acquiring the template image. In concrete terms, the state of the object inserted into the apparatus before the start of the measurement is imaged by a stereo camera. Then a plurality of corresponding feature points is extracted out of the acquired set of images, and a set of coordinates of the acquired plurality of feature points is acquired. It is preferable that the feature points are extracted from the object portion out of the object images. The coordinates of the feature point are expressed by a coordinate system of which origin is the center point of the stereo camera, but any coordinate system can be used only if each point can be set in the coordinate system.

In step S3, a plurality of feature points acquired in step S2 is tracked, and a motion vector connecting the corresponding feature points, between an original frame and a frame generated after a predetermined frame, is calculated. The feature points may be determined from one frame, or may be determined using a center of gravity of the feature point in a plurality of frames.

Whether the shift of the object is within the threshold or not is determined (step S4) using a motion vector calculated for each feature point. In this embodiment, a feature point of which moving distance is greatest is specified, and this moving distance is compared with the threshold, but a different method may be used. For example, an average value of the moving distances of all the feature points between two frames is determined, and this average value is compared with a threshold, or an integrated value of the moving distances of all the feature points since the start of the measurement is determined, and this value is compared with a threshold. Any method can be used for determining whether a position shift occurred.

If it is determined that a position shift occurred, whether the object moved in parallel or is deformed can be further estimated using a random sample consensus (RANSAC) method. In a RANSAC method, n number of feature points are randomly extracted, and a transformation matrix between the corresponding feature points is determined.

Then this transformation matrix is applied to other features points that are randomly extracted. If a significant number of transformation matrices of which square sum of residuals is the minimum are obtained as a result, then it can be determined that a position shift by parallel movement occurred. If such transformation matrices are not many, on the other hand, it can be determined that a rotation or deformation occurred. Needless to say, a method which is different from the above mentioned method may be used.

The processing operations in steps S41 and S5 to S7 are the same as Embodiment 2.

In step S8, notification to an operator is performed by a voice, a lamp, a screen display or the like, just like Embodiment 1 and 2, but how the object was shifted may be specifically notified as well. For example, the specific content may be notified by different colors, such as green lamp meaning that a shift did not occur, a yellow lamp meaning that a parallel movement did occur, and a red lamp meaning that a rotation or deformation did occur.

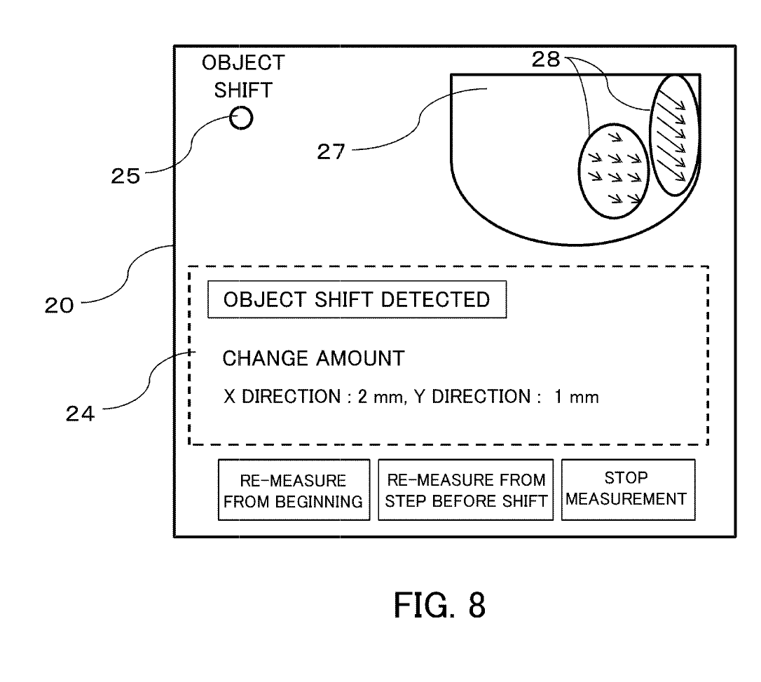

A graphic to indicate a motion vector of each feature point may be generated, and superimposed and displayed on the object image. Thereby details on a position change of the object can be notified to the operator. FIG. 8 is a screen example when a graphic to indicate the motion vectors of feature points (reference number 28) is generated, and superimposed and displayed on the object image 27. Here the feature points having similar motion vectors are clustered and displayed. Thereby how the object deformed can be clearly displayed to the operator.

The motion vectors may be displayed by a method other than the method of the above example. For example, similar motion vectors may be displayed with similar colors, or the color of lines may be changed when these lines are clustered. The motion vector may be displayed by a symbol other than an arrow, or only a region where a motion vector is large may be enlarged and displayed, without displaying the entire object.

In the photoacoustic measurement apparatus according to Embodiment 3, the feature points are extracted and motion vectors are calculated, whereby such a case of a part of the object being deformed can be supported, and how the object shifted can be accurately notified to the operator.

### MODIFICATION

The description of the embodiments is merely examples used for describing the present invention, and various changes and combination thereof are possible to carry out the invention without departing from the true spirit of the invention. The present invention can also be carried out as a control method for an object information acquiring apparatus that includes at least a part of the above mentioned processing. The above mentioned processing and means can be freely combined to carry out the invention as long as there is no technical inconsistency generated.

For example, in the description of the embodiments, an example of matching the patterns of the object images and an example of comparing the coordinates of the feature points were used, but other information may be used for detecting a position shift of the object. For example, a background portion of the object, inter-frame difference information, inter-frame difference information from a frame after a predetermined time, histogram information of the object portion, texture information of the object, or optical flow information based on a gradient method or block matching method can be used.

Information based on a mobile object tracking method using a Moravec operator, a Kanade-Lucas-Tomasi (KLT) method, a local correlation correspondence method or a method that considers global consistency may be used. It may be simply determined that a position shift occurred if the object protrudes from the predetermined area. Generation of a position shift may be determined by any information as long as the change of position or outer shape of the object can be known by the information.

A value that is set as a threshold and a value displayed to the operator may be the following examples beside a number of pixels changed from the initial state as used for the embodiments. For example, a displacement amount of each feature point between frames, an integrated value of a displacement amount of each feature point within a predetermined time, a change direction of each feature point in a space, acquisition change data converted into a voxel value, or an mm or cm value may be used.

The result of the shift amount classified into large, intermediate and small, the type of position shift (e.g. “parallel moving”, “partial distortion”), a shift value from the initial state on each axis of the coordinate system or the like may also be used for the shift amount. Any value may be used only if the state of the object position shift can be expressed.

In the above embodiments, the photoacoustic measurement apparatus was described as an example, but the present invention may be applied to an ultrasonic measurement apparatus that includes an acoustic wave transmission unit to transmit an ultrasound wave to an object and visualize information related to acoustic characteristics inside the object by receiving the ultrasound value reflected inside the object. The present invention can be applied to any apparatus that acquires information inside an object by receiving an acoustic wave which arrives from an interior of the object.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-086694, filed on Apr. 17, 2013, which is hereby incorporated by reference herein in its entirety.

What is claimed is:

1. An object information acquiring apparatus comprising:
an acoustic wave probe that receives an acoustic wave arriving from an interior of an object;
a position information acquisition unit that acquires position information which is information on a position of the object; and
a notification unit that notifies an operator of a change in the position of the object, based on the position information.

2. The object information acquiring apparatus according to claim 1, wherein
the notification unit acquires a moving amount of an object based on the position information, and notifies the operator when the moving amount exceeds a predetermined value.

3. The object information acquiring apparatus according to claim 2, wherein
the position information acquisition unit is a camera that captures an image of an object,
the position information is an object image captured by the camera, and
the notification unit acquires a moving amount of the object by performing pattern matching of a plurality of object images captured at different timings.

4. The object information acquiring apparatus according to claim 1, wherein
the notification unit acquires a deformation amount of an object based on the position information, and notifies the operator when the deformation amount exceeds a predetermined value.

5. The object information acquiring apparatus according to claim 4, wherein
the position information acquisition unit is a camera that captures an image of an object;
the position information is an object image captured by the camera, and
the notification unit acquires a deformation amount of the object by extracting one or more feature points respectively from a plurality of object images captured at different timings, and detecting a change of coordinates of each extracted feature point.

6. The object information acquiring apparatus according to claim 1, wherein
the notification unit generates and outputs an image that indicates movement of the object.

7. The object information acquiring apparatus according to claim 1, further comprising:
a light irradiation unit that irradiates light onto the object; and
an image acquisition unit that images information related to optical characteristics inside the object by analyzing an acoustic wave generated inside the object due to light.

8. The object information acquiring apparatus according to claim 1, further comprising:
an acoustic wave transmission unit that transmits an acoustic wave into the object in use of the acoustic wave probe; and
an image acquisition unit that images information related to the acoustic characteristics inside the object by analyzing an acoustic wave reflected inside the object.

9. A control method for an object information acquiring apparatus having an acoustic wave probe that receives an acoustic wave arriving from an interior of an object,
the method comprising:
a reception step of receiving an acoustic wave in use of the acoustic wave probe;
a position information acquisition step of acquiring position information which is information on a position of the object; and
a notification step of notifying an operator of a change in the position of the object, based on the position information.

10. The control method for an object information acquiring apparatus according to claim 9, wherein
in the notification step, a moving amount of an object is acquired based on the position information, and the operator is notified when the moving amount exceeds a predetermined value.

11. The control method for an object information acquiring apparatus according to claim 10, wherein
in the position information acquisition step, an image of the object is captured using a camera,
the position information is an object image captured by the camera, and
in the notification step, a moving amount of the object is acquired by performing pattern matching of a plurality of object images captured at different timings.

12. The control method for an object information acquiring apparatus according to claim 9, wherein
in the notification step, a deformation amount of an object is acquired based on the position information, and the operator is notified when the deformation amount exceeds a predetermined value.

13. The control method for an object information acquiring apparatus according to claim 12, wherein
in the position information acquisition step, an image of the object is captured using a camera,
the position information is an object image captured by the camera, and
in the notification step, a deformation amount of the object is acquired by extracting one or more feature points respectively from a plurality of object images captured at different timings, and detecting a change of coordinates of each extracted feature point.

14. The control method for an object information acquiring apparatus according to claim 9, wherein
in the notification step, an image that indicates movement of the object is generated and outputted.

15. The control method for an object information acquiring apparatus having a light irradiation unit that irradiates light onto an object according to claim 9,
the method further comprising:
a light irradiation step of generating light from the light irradiation unit; and
an image acquisition step of imaging information related to optical characteristics inside the object by analyzing an acoustic wave generated inside the object due to the light.

16. The control method for an object information acquiring apparatus, the acoustic wave probe of which has a function of transmitting an acoustic wave into an object, according to claim 9,
the method further comprising:
an acoustic wave transmission step of transmitting an acoustic wave from the acoustic wave probe; and
an image acquisition step of imaging information related to the acoustic characteristics inside the object by analyzing an acoustic wave reflected inside the object.
